(19) United States
(12) Patent Application Publication
(10) Pub. No.: US 2013/0171159 A1
(43) Pub. Date: Jul. 4, 2013

(54) PHOSPHORYLATED TWIST1 AND METASTASIS

(71) Applicants: Brian Arthur Hemmings, Bettingen (CH); Gongda Xue, Allschwill (CH)

(72) Inventors: Brian Arthur Hemmings, Bettingen (CH); Gongda Xue, Allschwill (CH)

(21) Appl. No.: 13/821,373

(22) PCT Filed: Sep. 9, 2011
(86) PCT No.: PCT/EP2011/065615

§ 371 Date: Mar. 7, 2013

(30) Foreign Application Priority Data
Sep. 10, 2010 (EP) 10176297.9

(51) Int. Cl. A61K 39/395; A61K 38/00

(52) U.S. Cl. CPC A61K 39/3955; A61K 38/00
USPC 424/139.1; 514/19.3; 435/7.1

(57) ABSTRACT

The present application relates to methods for metastasis in a subject by modulating the phosphorylation of the Serine 42 of Twist1 by administering to said subject a therapeutically effective amount of a modulator of said phosphorylation of the Serine 42 of Twist1. Antibodies, uses methods and biomarkers based on the

[symbol]

### FIELD OF THE INVENTION

The present invention relates to a method of treating cancer metastasis by modulating the phosphorylation of the serine 42 of Twist-1.

### BACKGROUND OF THE INVENTION

Cancer metastasis is the major cause of death in human cancer (Weigelt et al., 2005). Metastasis of epithelial tumors, which comprise of almost 90% of all cancers, occurs when tumor cells overcome fundamental cell controls maintaining cell-cell contacts and preventing migration. Accumulating experimental data indicate that these controls in epithelial tumor cells are overcome due to the activation of a dormant developmental process, EMT, when epithelial cells transform into mesenchymal cells and migrate to areas where they form bone, muscle, connective tissue and blood vessels (Garber, 2008). The occurrence of EMT in situ was also illustrated recently in both mice and primary human breast cancer specimens (Trimboli et al., 2008). Activation of the EMT gene program that promotes migration in physiological conditions or metastasis in tumors has been reported for several transcription factors including Twist, Snail and Zeb (Polyak and Weinberg, 2009).

The evolutionarily conserved basic helix-loop-helix (bHLH) transcription factor Twist plays an important role in morphogenesis during embryonic development by regulating cell migration (Castanon and Baylies, 2002). In the developing embryo, expression of Twist proteins is most frequently observed in mesoderm and ectoderm and Twist1 knockout mice die at about E11.5 due to closure of the neural tube, specifically in the cranial region (Chen and Behringer, 1995). In the pathological setting of cancer, Twist1 over expression is found in a variety of tumors (Ansieau et al., 2008) and correlates with poor prognosis, tumor progression and metastasis. In a study examining 54 human invasive breast carcinoma samples, Twist expression was elevated in 70% of invasive lobular carcinomas (ILC) (Yang et al., 2004a). In mice, Twist1 expression is tightly associated with progression of breast tumors and is essential for metastasis from the mammary gland to the lung (Yang et al., 2004a). In cells, Twist induces EMT by downregulating E-cadherin, catenin proteins that are crucial for maintaining the integrity of epithelial cell contacts and promotes cell migration. In hepatocelluar carcinomas, overexpression of Twist correlates with EMT-associated changes and metastasis (Lee et al., 2006b) and Twist1-mediated EMT in hepatocellular carcinomas is also associated with the transcriptional suppression of E-cadherin levels (Vesuna et al., 2008). Interestingly, lowered E-cadherin levels in cells promote metastasis by impairing its intracellular signaling and inducing Twist (Onder et al., 2008). Overexpression of Twist negatively regulates programmed cell death (Puisieux et al., 2006) and overcome oncogene-induced senescence, indicating that Twist1 is linked to tumor progression. Twist1 haploinsufficiency induced calvarial osteoblast apoptosis and Twist1 homozygous knockout induced massive apoptosis during mammalian development (Chen and Behringer, 1995). In mouse embryonic fibroblasts (MEFs), Twist1 blocks the c-myc-induced apoptotic response (Maestro et al., 1999). A genome-wide microarray analysis of human neuroblastoma (NB) illustrated that N-Myc amplification is constantly associated with overexpression of Twist1 and knockdown of Twist1 in NB cells promoted apoptosis (Valsesia-Wittmann et al., 2004). Consistent with an anti-apoptotic role for Twist, ectopically expressed Twist1 in non N-Myc amplified NB cells inhibited the p53 response to genotoxic stress. Furthermore, Twist1 interacts with HOXA5, a potent transactivator of the p53 promoter, thereby modulating its activity (Stasinopoulos et al., 2005). Finally, Twist proteins were able to override oncogene-induced premature senescence in a mouse model (Ansieau et al., 2008). These studies indicate that Twist1 can play dual roles in the regulation of anti-apoptosis and metastasis during tumor progression.

### SUMMARY OF THE INVENTION

The present inventors noted that though the growth of primary tumor in the mammary gland is not influenced by this modification, S42 phosphorylation of Twist1 is essential for metastasis of breast tumor cells to the lung and the establishment of secondary tumor growth at distal sites. Collectively, the inventors' data indicate that the Akt signaling pathway can directly influence EMT and the invasiveness of cancer cells by phosphorylation of Twist1, thus uncovering a novel mechanism linking the PI3K/Akt pathway to tumor progression and malignancy.

The present invention thus encompasses a method for inhibiting metastasis in a subject by modulating the phosphorylation of the Serine 42 of Twist1 by administering to said subject a therapeutically effective amount of a modulator of said phosphorylation of the Serine 42 of Twist1. In some embodiments, the phosphorylation of the Serine 42 of Twist1 is modulated by an inhibitor which specifically binds to Twist1 and hinders the phosphorylation of its Serine 42 by PKB, for instance an antibody or a small molecule.

In some embodiments of the invention, the subject is a mammal, for instance a human subject. In some embodiments, the method of the invention is performed in vivo, ex vivo or in vitro.

In some embodiments of the invention, the epithelial-mesenchymal transition (EMT) of cancer cells is reduced. In some embodiments, the cancer is a melanoma, a colorectal cancer, a breast cancer, a lung cancer or a prostate cancer.

The present invention also encompasses an antibody or a small molecule specifically binding to Twist1 and hindering the phosphorylation of the Serine 42 of Twist1 by PKB, for use as a medicament inhibit metastasis, for instance, said antibody specifically binds to an epitope of Twist1, which epitope comprises the Serine 42 of Twist1.

In some embodiments, fragments of the Twist1 protein, which fragments comprise an amino acid corresponding to the Serine 42 of Twist1 and is recognized and phosphorylated by PKB, can be used as a medicament inhibit metastasis by modulating the phosphorylation of the Serine 42 of Twist1 by PKB.

The present invention also encompasses a method for the identification of a substance that modulates a PKB signaling pathway and is a potential medicament inhibit metastasis, which method comprises the step of assessing the phosphorylation of the Serine 42 of Twist1.

Another aspect of the present invention is a method of diagnosing metastasis comprising the step of assessing the phosphorylation of the Serine 42 of Twist1. In some embodiments, an increased phosphorylation of the Serine 42 of Twist1 is indicative of metastasis.

A further aspect of the present invention encompasses the use of the phosphorylation of the Serine 42 of Twist1 as a biomarker for metastasis to stratify cancer patients.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1: Twist1 is Phosphorylated on S42 by Activated Akt and Correlates with Enhanced Akt Phosphorylation.

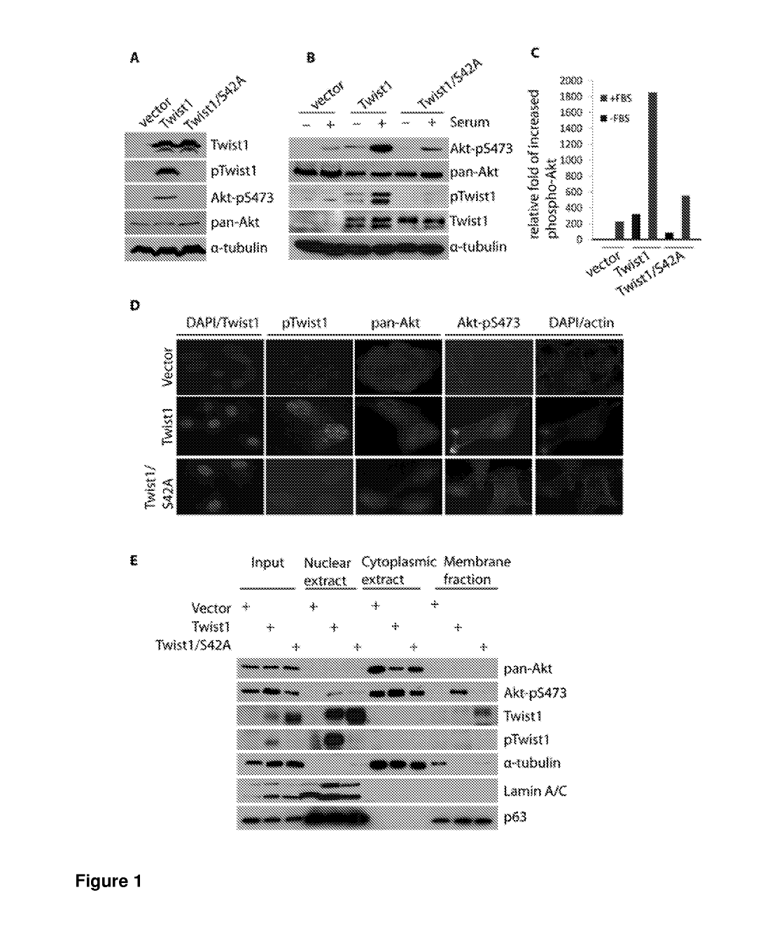

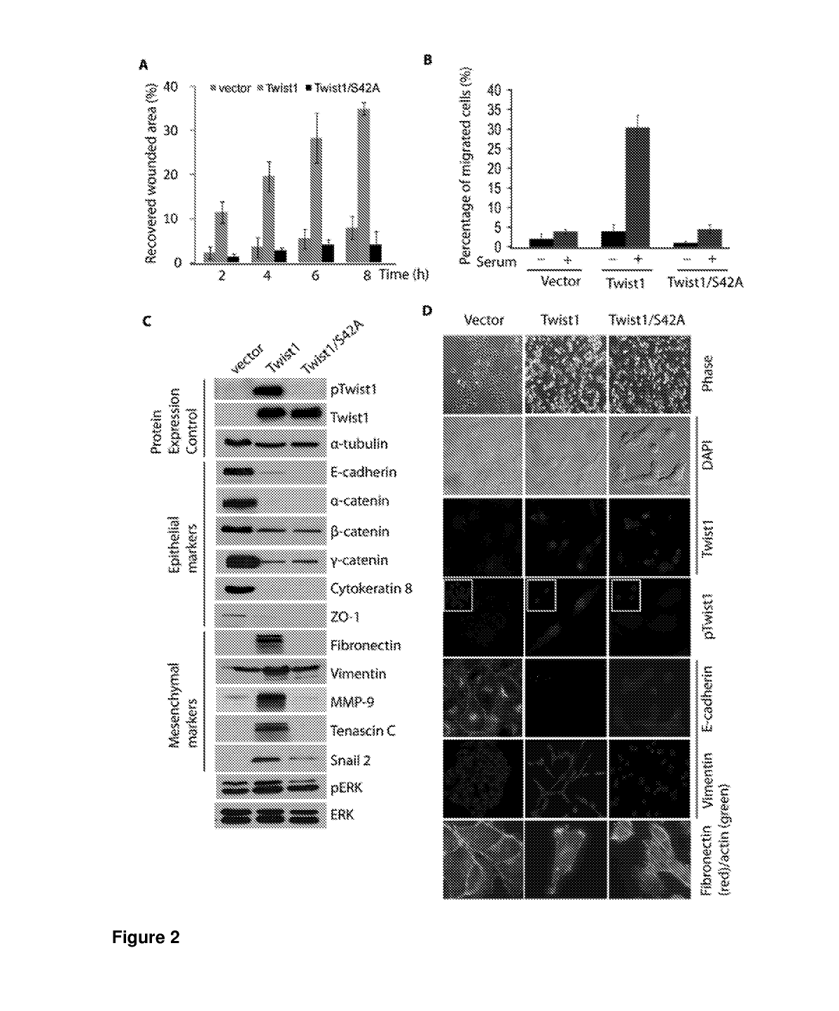

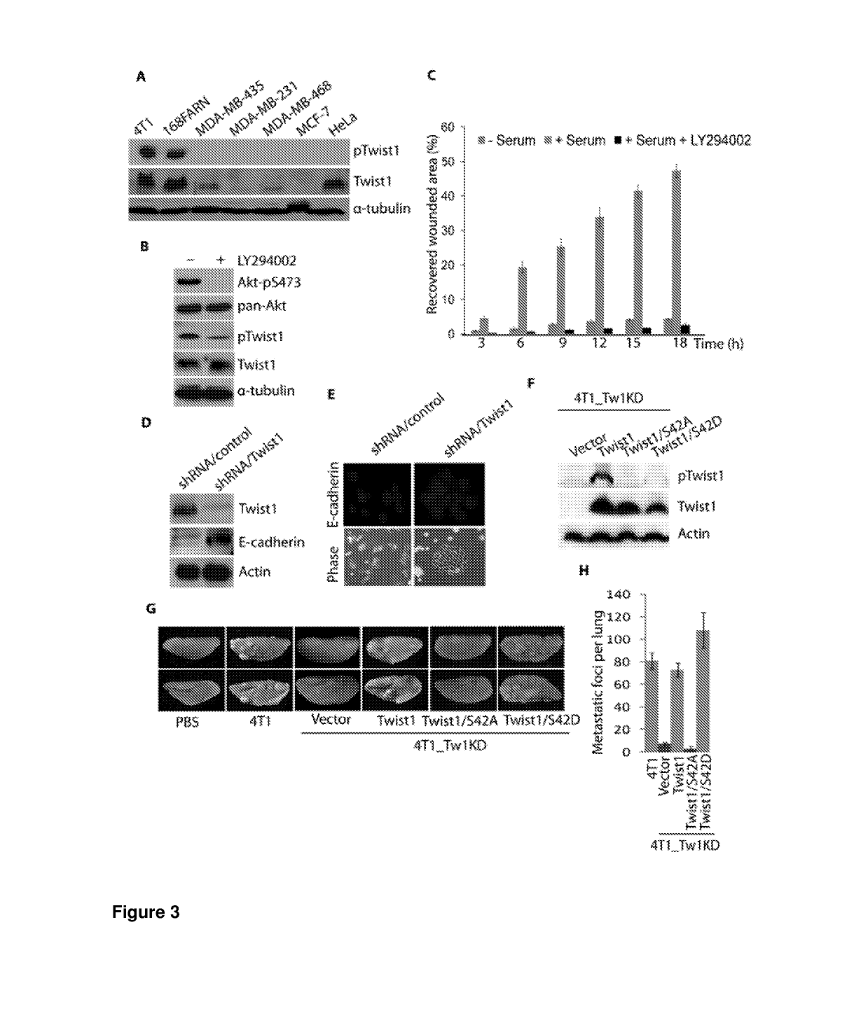

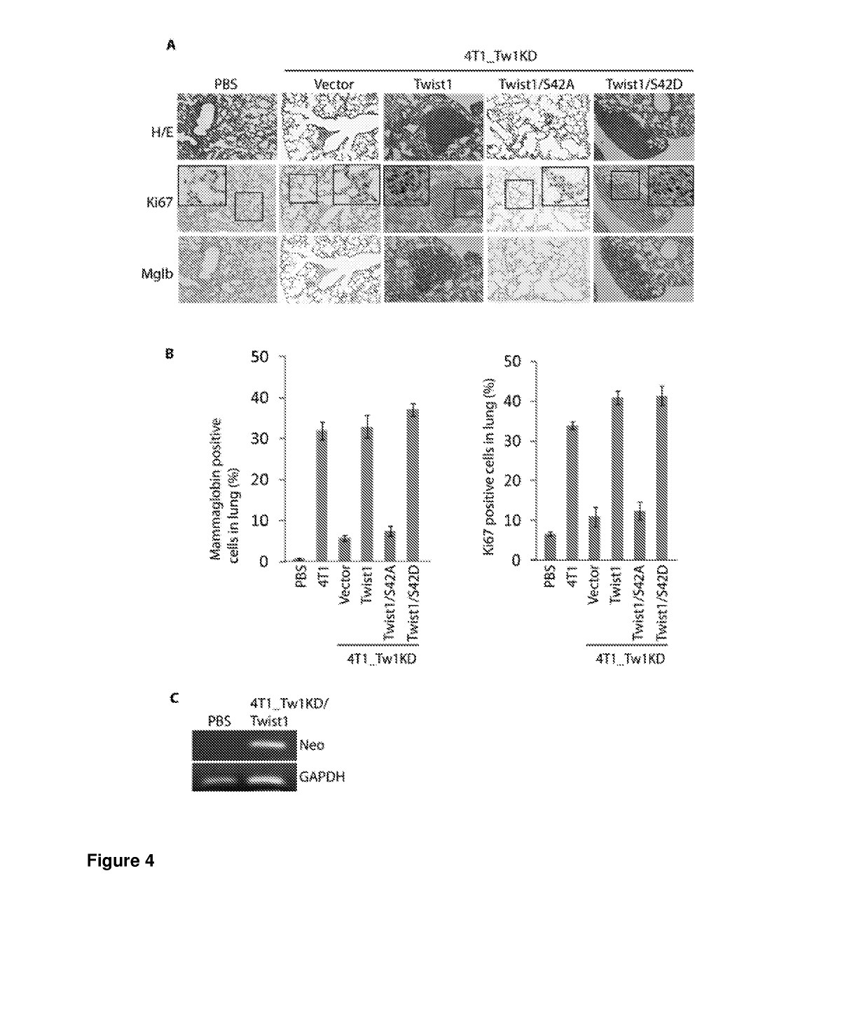

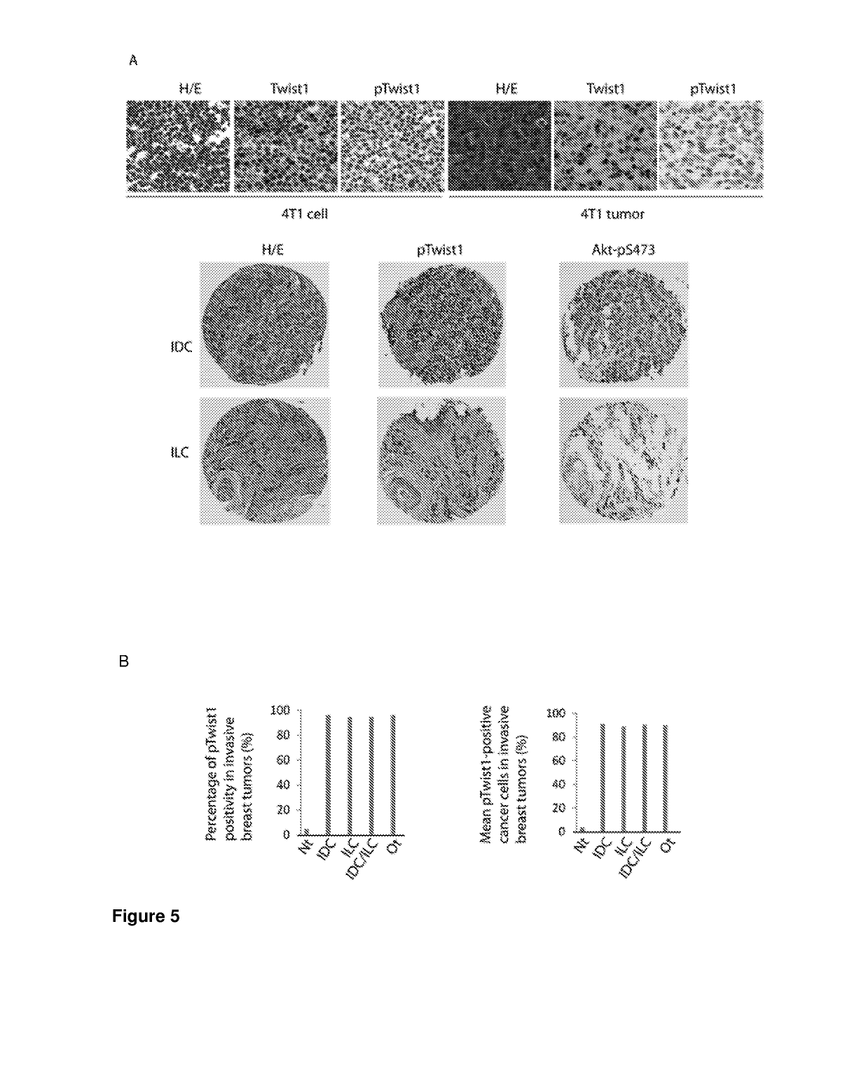

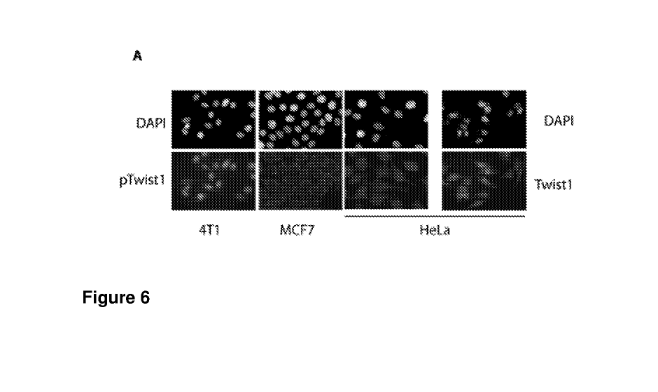

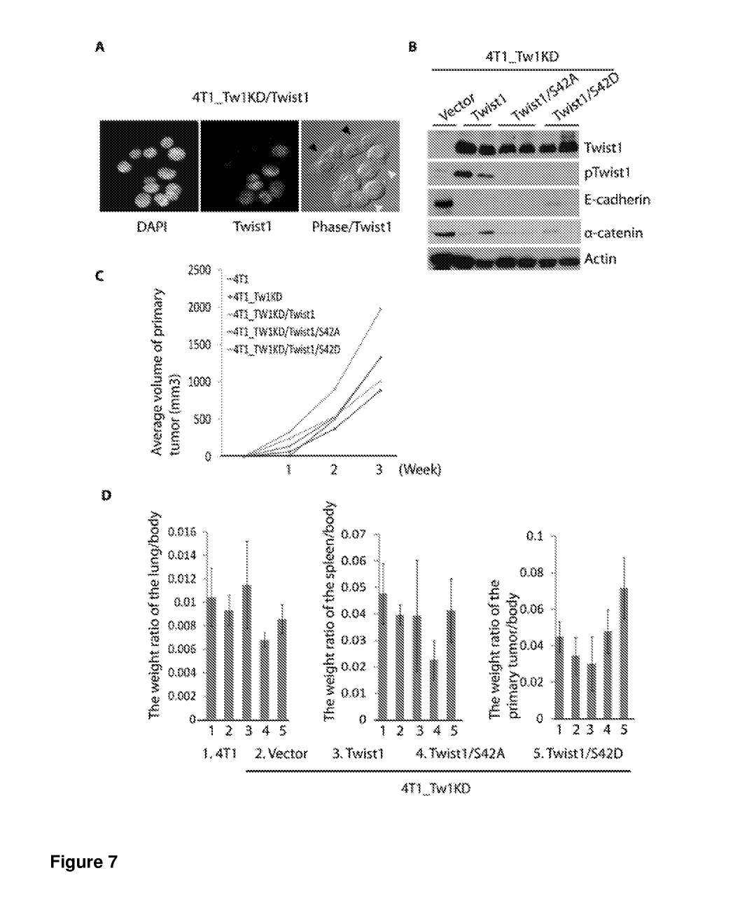

Wild-type Twist1 and mutant Twist1/S42A are stably expressed in MDCK cells. (A) Expression and phosphorylation analysis of Twist1 and total Akt in crude MDCK lysates. MDCK cells transfected with empty plasmid was used as control. (B) MDCK cells expressing Twist1 and Twist1/S42A were serum-starved overnight and stimulated with 10% FBS for 1 h. The crude lysates were subjected to western blotting analysis for Twist1 and Akt phosphorylation. (C) Quantitative analysis of Akt phosphorylation induced by Twist1 expression. (D) Immunofluorescence staining of Twist1, pTwist1, pan-Akt, Akt-pS473 and stress fibers in MDCK cells that stably express Twist1 and Twist1/S42A. (E) Subcellular fractionation analysis of the cellular localization of pTwist1 and Akt-pS473. Lamin A/C, α-Tubulin and p63 were used as controls of nuclear fraction, cytoplasmic fraction and membrane fraction, respectively.

FIG. 2: Phosphorylation of Twist1 Triggers EMT Associated with Increased Cell Migration and Invasion.

(A) Wound healing assay of MDCK cells expressing wild-type Twist1 or mutant Twist1/S42A. The cells were seeded in Ibidi chambers and starved overnight. Cell migration was recorded by live-imaging microcopy and the migratory potential is presented as size of uncovered area over the initial wounded area (calculated with Metamorph). Bars represent means±SEM of samples measured in triplicate. (B) The invasion ability was elevated toward serum. MDCK cells expressing empty vector (negative control), wild-type Twist1 and Twist1/S42A were loaded into Boyden-chambers and the invasion potential presented as percentage of migrating cells over total cell numbers. Bars represent means±SEM of samples measured in triplicate. (C) Analysis of expression of epithelial markers including E-cadherin, α-catenin, β-catenin, γ-catenin, cytokeratin 8, ZO-1, and mesenchymal markers including fibronectin, vimentin, MMP-9, tenascin C and Snail 2 was determined by immunoblotting. Correlated Twist1 phosphorylation and increased ERK phosphorylation are also shown. α-Tubulin was used as a loading control. (D) The morphologies of the MDCK cells expressing either the empty vector, wild-type Twist1 or Twist1/S42A mutant revealed by phase contrast microscopy and immunofluorescence staining of phosphorylated Twist1, E-cadherin, vimentin and fibronectin in the same cells.

FIG. 3: Phosphorylation of Twist1 is Essential for Breast Tumor Metastasis from the Mammary Gland to the Lung.

(A) Analysis of Twist1 phosphorylation in several breast cancer cell lines. α-Tubulin was used as a loading control. (B) 4T1 cells were treated with 50 nM LY294002 and the phosphorylation status of Twist1 and Akt was analyzed by immunoblotting. (C) 4T1 cells were starved overnight and pre-treated with 20 nM LY294002 for 2 h. The cells were then incubated with serum-free medium, serum-containing medium or serum-containing medium with 20 nM LY294002. Cell migration was recorded by live-imaging microscopy for 20 h. The data were analyzed with Metamorph. Bars represent means±SEM of samples measured in triplicate. (D) Twist1 was knocked down in 4T1 cells. (E) Immunofluorescence staining of E-cadherin in Twist1-knockdown 4T1 cells; the corresponding cell morphologies were revealed by phase contrast microscopy. (F) Immunoblotting analysis of the phosphorylation status of ectopically expressed Twist1 and its variants in 4T1_Tw1KD cells. (G) Lung metastases of BALB/c mice injected with 4T1_Tw1KD cells expressing Twist1 variants. The lung tissue was stained in Bouin solution and revealed by MacroFluo Z6 (Leica, Germany) for both sides of the large lobe. (H) Total numbers of visible metastatic nodules in lungs of individual mice carrying mammary tumors developing from 4T1_Tw1KD cells expressing Twist1 variants at 22 days post-tumor implantation. Each group included 6 mice. Error bars represent SEM.

FIG. 4: Immunohistochemical Staining of Lung Metastases.

Lung tissue was fixed in PFA overnight and embedded in paraffin. Staining was carried out with Ventana Discovery X. (A) Serial sections of lung tissue were stained for Ki67 and mammaglobin and with H/E. (B) Statistic analysis of mammaglobin- or Ki67-positive cells in individual serial sections of lungs. Bar represent means±SEM of samples measured in triplicate. (C) The gene encoding the Neomycin-cleaving enzyme was examined in lungs by PCR. The GAPDH gene was used as an internal control.

FIG. 5: Twist1 is Phosphorylated in Invasive Human Breast Cancers.

Primary breast tumors dissected from xenograft mice were fixed in PFA and embedded in paraffin. Injected 4T1 cells were prepared in the same way as a control. Human breast tumor samples from University hospital, Basel and were processed via TMA at the Institute of Pathology. Out of 2094 samples, 1532 were evaluable. (A) Immunohistochemical staining of 4T1 cells and primary tumors from injected BALB/c mice with total Twist1 and phosphor-Twist1 antibodies and H/E. In addition, invasive human breast tumors including IDC, ILC types were stained with phospho-Twist1 and phospho-Akt antibodies and with H/E. (B) Pathological statistics of 1532 clinically diagnosed invasive human breast tumors of IDC, ILC, IDC/ILC types staining positive for phosphor-Twist1 (Nt: Non-tumor; Ot: Other types). The cut-off was set to 30% positive cells.

FIG. 6: Endogenous Twist1 is Phosphorylated in Invasive Cancer Cells.

Phosphorylated Twist1 was detected in invasive 4T1 cells by immunofluorescence staining. For comparison, Twist1 expression and phosphorylation were not detected in MCF 7. Twist1 expression was detected in HeLa cells but was not phosphorylated.

FIG. 7: Phosphorylation of Twist1 Specifically Regulates Breast Cancer Metastasis In Vivo.

(A) Twist1 and its variants were expressed in 4T1_Tw1KD cells and Twist1 expression and cell-cell contact were analysed by immunofluorescence staining. Black arrows indicate the cell-cell contact in 4T1_Tw1KD cells that did not express Twist1 and yellow arrows indicate the disrupted junction area in 4T1_Tw1KD cells expressing Twist1. (B) Twist1 expression and phosphorylation analysis in 4T1_Tw1KD cells expressing Twist1 and its variants. (C) The size of primary tumors measured weekly with calipers. A total of 6 mice were examined in each group. (D) Analysis of the ratio of lung, spleen and primary tumors to total body weight of BALB/c mice injected with 4T1_Tw1KD cells expressing Twist1 and its variants.

### DETAILED DESCRIPTION OF THE INVENTION

The present inventors noted that though the growth of primary tumor in the mammary gland is not influenced by this modification, S42 phosphorylation of Twist1 is essential for metastasis of breast tumor cells to the lung and the establishment of secondary tumor growth at distal sites. Collectively, the inventors' data indicate that the Akt signaling pathway can directly influence EMT and the invasiveness of cancer cells by phosphorylation of Twist1, thus uncovering a novel mechanism linking the PI3K/Akt pathway to tumor progression and malignancy.

The present invention thus encompasses a method for inhibiting metastasis in a subject by modulating the phosphorylation of the Serine 42 of Twist1 by administering to said subject a therapeutically effective amount of a modulator of said phosphorylation of the Serine 42 of Twist1. In some embodiments, the phosphorylation of the Serine 42 of Twist1 is modulated by an inhibitor which specifically binds to Twist1 and hinders the phosphorylation of its Serine 42 by PKB, for instance an antibody or a small molecule.

In some embodiments of the invention, the subject is a mammal, for instance a human subject. In some embodiments, the method of the invention is performed in vivo, ex vivo or in vitro.

In some embodiments of the invention, the epithelial-mesenchymal transition (EMT) of cancer cells is reduced. In some embodiments, the cancer is a melanoma, a colorectal cancer, a breast cancer, a lung cancer or a prostate cancer.

The present invention also encompasses an antibody or a small molecule specifically binding to Twist1 and hindering the phosphorylation of the Serine 42 of Twist1 by PKB, for use as a medicament inhibit metastasis, for instance, said antibody specifically binds to an epitope of Twist1, which epitope comprises the Serine 42 of Twist1.

In some embodiments, fragments of the Twist1 protein, which fragments comprise an amino acid corresponding to the Serine 42 of Twist1 and is recognized and phosphorylated by PKB, can be used as a medicament inhibit metastasis by modulating the phosphorylation of the Serine 42 of Twist1 by PKB.

The present invention also encompasses a method for the identification of a substance that modulates a PKB signaling pathway and is a potential medicament inhibit metastasis, which method comprises the step of assessing the phosphorylation of the Serine 42 of Twist1.

Another aspect of the present invention is a method of diagnosing metastasis comprising the step of assessing the phosphorylation of the Serine 42 of Twist1. In some embodiments, an increased phosphorylation of the Serine 42 of Twist1 is indicative of metastasis.

A further aspect of the present invention encompasses the use of the phosphorylation of the Serine 42 of Twist1 as a biomarker for metastasis to stratify cancer patients.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=1, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

In one embodiment where one is assaying for the ability to bind or compete with full-length Twist-1 polypeptide for binding to anti-phosphorylated serine 42 of Twist-1 antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Assays described herein and otherwise known in the art may routinely be applied to measure the ability of polypeptides and variants derivatives and analogs thereof, comprising at least 8 amino acids of Twist-1, wherein one of said amino acids corresponds to the serine 42 of Twist-1, to elicit Twist-1-related biological activity (either in vitro or in vivo) and/or to assess whether Twist-1 is present in a given sample, e.g. a sample isolated from a patient.

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834, 252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In addition, in the context of the present invention, the term “antibody” shall also encompass alternative molecules having the same function, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues.

Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571, 698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716, 111; and PCT publications WO 98/46645, WO 98/50433, WO 98/24893, WO 98/16654, WO 96/34096, WO 96/33735, and WO 91/10741. Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569, 825; 5, 661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity.

Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)). The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

By “affinity” as used herein is meant the propensity of one chemical species to separate or dissociate reversibly from another chemical species. In the present invention, the two chemical species most typically are represented by a protein and its ligand, more specifically an antibody and its target antigen. Affinity herein is measured by the equilibrium constant of dissociation (Kd or K_{d}) that defines the binding between the two chemical species. The Kd defines how tightly the species bind one another. The smaller the dissociation constant, the more tightly bound the ligand is, or the higher the affinity between ligand and protein. For example, an antigen with a nanomolar (nM) dissociation constant binds more tightly to a particular antibody than a ligand with a micromolar (μM) dissociation constant. By “greater affinity” or “improved affinity” or “enhanced affinity” or “better affinity” than a parent polypeptide, as used herein is meant that a protein variant binds to its ligand with a significantly higher equilibrium constant of association (KA or Kₐ) or lower equilibrium constant of dissociation (Kd or K_{d}) than the parent protein when the amounts of variant and parent polypeptide in the binding assay are essentially the same. For example, in the context of antibodies, a variant antibody may have greater affinity to the antigen that its parent antibody, for example when the CDRs are humanized, as described herein. Alternatively, and Fc polypeptide may have greater affinity to an Fc receptor, for example, when the Fc variant has greater affinity to one or more Fc receptors or the FcRn receptor. In general, the binding affinity is determined, for example, by binding methods well known in the art, including but not limited to Biacore™ assays. Accordingly, by “reduced affinity” as compared to a parent protein as used herein is meant that a protein variant binds its ligand with significantly lower Ka or higher Kd than the parent protein. Again, in the context of antibodies, this can be either to the target antigen, or to a receptor such as an Fc receptor. Greater or reduced affinity can also be defined relative to an absolute level of affinity. For example, greater or enhanced affinity may mean having a Kd lower than about 10 nM, for example between about 1 nM-about 10 nM, between about 0.1-about 10 nM, or less than about 0.1 nM.

The term “specifically binds” refers, with respect to an antigen to the preferential association of an antibody or other ligand, in whole or part, with a cell or tissue bearing that antigen and not to cells or tissues lacking that antigen. It is recognized that a certain degree of non-specific interaction can occur between a molecule and a non-target cell or tissue. Nevertheless, specific binding can be distinguished as mediated through specific recognition of the antigen. Although selectively reactive antibodies bind antigen, they can do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a specific antigen as compared to an unspecific antigen. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

The present invention is also directed to antibody-based therapies which involve administering antibodies of the invention to an animal, in some embodiments, a mammal, for example a human, patient to treat cancer. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating cancer in a subject by inhibiting the phosphorylation of the serine 42 of Twist-1 by administration to the subject of an effective amount of an inhibitory compound or pharmaceutical composition comprising such inhibitory compound. In some embodiments, said inhibitory compound is an antibody. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of cancer by inhibiting the phosphorylation of the serine 42 of Twist-1. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. In some embodiments, the dosage administered to a patient is between 0.1 mg/kg and 20 mg/kg of the patients body weight, for example 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown' of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein. In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

The inhibitors of the phosphorylation of the serine 42 of Twist-1 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The inhibitors of the phosphorylation of the serine 42 of Twist-1 may be used in a number of ways. For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted at the site of a tumour, and the molecule may be released over weeks or months. Such devices may be particularly advantageous when long term treatment with an inhibitor of the phosphorylation of the serine 42 of Twist-1 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an inhibitor of the phosphorylation of the serine 42 of Twist-1 may be used for the treatment of cancer in the subject, depending upon which specific inhibitor is used. When the inhibitor is delivered to a cell, daily doses may be given as a single administration (e.g. a single daily injection).

Various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term “cancer” refers to a group of diseases in which cells are aggressive (grow and divide without respect to normal limits), invasive (invade and destroy adjacent tissues), and sometimes metastatic (spread to other locations in the body). These three malignant properties of cancers differentiate them from benign tumors, which are self-limited in their growth and don't invade or metastasize (although some benign tumor types are capable of becoming malignant). A particular type of cancer is a cancer forming solid tumours. Such cancer forming solid tumours can be breast cancer, prostate carcinoma or oral squamous carcinoma. Other cancer forming solid tumours for which the methods and inhibitors of the invention would be well suited can be selected from the group consisting of adrenal cortical carcinomas, angiomatoid fibrous histiocytomas (AFH), squamous cell bladder carcinomas, urothelial carcinomas, bone tumours, e.g. adamantinomas, aneurysmal bone cysts, chondroblastomas, chondromas, chondromyxoid fibromas, chondrosarcomas, fibrous dysplasias of the bone, giant cell tumours, osteochondromas or osteosarcomas, breast tumours, e.g. secretory ductal carcinomas, chordomas, clear cell hidradenomas of the skin (CCH), colorectal adenocarcinomas, carcinomas of the gallbladder and extrahepatic bile ducts, combined hepatocellular and cholangiocarcinomas, fibrogenesis imperfecta ossium, pleomorphic salivary gland adenomas head and neck squamous cell carcinomas, chromophobe renal cell carcinomas, clear cell renal cell carcinomas, nephroblastomas (Wilms tumor), papillary renal cell carcinomas, primary renal ASPSCR1-TFE3 t(X;17)(p11;q25) tumors, renal cell carcinomas, laryngeal squamous cell carcinomas, liver adenomas, hepatoblastomas, hepatocellular carcinomas, non-small cell lung carcinomas, small cell lung cancers, malignant melanoma of soft parts, medulloblastomas, meningiomas, neuroblastomas, astrocytic tumours, ependymomas, peripheral nerve sheath tumours, neuroendocrine tumours, e.g. phaeochromocytomas, neurofibromas, oral squamous cell carcinomas, ovarian tumours, e.g. epithelial ovarian tumours, germ cell tumours or sex cord-stromal tumours, pericytomas, pituitary adenomas, posterior uveal melanomas, rhabdoid tumours, skin melanomas, cutaneous benign fibrous histiocytomas, intravenous leiomyomatosis, aggressive angiomyxomas, liposarcomas, myxoid liposarcomas, low grade fibromyxoid sarcomas, soft tissue leiomyosarcomas, biphasic synovial sarcomas, soft tissue chondromas, alveolar soft part sarcomas, clear cell sarcomas, desmoplastic small round cell tumours, elastofibromas, Ewing's tumours, extraskeletal myxoid chondrosarcomas, inflammatory myofibroblastic tumours, lipoblastomas, lipoma, benign lipomatous tumours, liposarcomas, malignant lipomatous tumours, malignant myoepitheliomas, rhabdomyosarcomas, synovial sarcomas, squamous cell cancers, subungual exostosis, germ cell tumours in the testis, spermatocytic seminomas, anaplastic (undifferentiated) carcinomas, oncocytic tumours, papillary carcinomas, carcinomas of the cervix, endometrial carcinomas, leiomyoma as well as vulva and/or vagina tumours. In an embodiment of the invention, the cancer is a colorectal cancer, a breast cancer, a lung cancer or a prostate cancer.

As used herein, the term “metastasis” refers to the spread of cancer cells from one organ or body part to another area of the body, i.e. to the formation of metastases. This movement of tumor growth, i.e. metastasis or the formation of metastases, occurs as cancer cells break off the original tumor and spread e.g. by way of the blood or lymph system. Without wishing to be bound by theory, metastasis is an active process and involves an active breaking from the original tumor, for instance by protease digestion of membranes and or cellular matrices, transport to another site of the body, for instance in the blood circulation or in the lymphatic system, and active implantation at said other area of the body. In one embodiment, the cancer is a cancer dependent on the phosphorylation of the serine 42 of Twist-1. Cancers dependent on the phosphorylation of the serine 42 of Twist-1 are cancers where the phosphorylation of the serine 42 of Twist-1 has become essential. Cancers dependent on the phosphorylation of the serine 42 of Twist-1 can be easily identified by inhibiting the phosphorylation of the serine 42 of Twist-1, and identifying the cancers that are not able to grow, migrate or forming metastases in the absence of it.

The present invention also provides a method of screening compounds to identify those which might be useful for treating cancer in a subject by inhibiting the phosphorylation of the serine 42 of Twist-1 as well as the so-identified compounds.

As used herein and as in the fields of pharmacology and biochemistry, a “small molecule” is a low molecular weight organic compound which is by definition not a polymer. The term small molecule is restricted to a molecule that also binds with high affinity to a biopolymer such as protein, nucleic acid, or polysaccharide and in addition alters the activity or function of the biopolymer. The upper molecular weight limit for a small molecule is approximately 800 Daltons which allows for the possibility rapid diffuse across cell membranes so that they can reach intracellular sites of action. In addition, this molecular weight cutoff is necessary but insufficient condition for oral bioavailability. Small molecules can have a variety of biological functions, serving as cell signalling molecules, as tools in molecular biology, as drugs in medicine, and in countless other roles. These compounds can be natural (such as secondary metabolites) or artificial (such as antiviral drugs); they may have a beneficial effect against a disease (such as drugs) or may be detrimental (such as teratogens and carcinogens). Biopolymers such as nucleic acids, proteins, and polysaccharides (such as starch or cellulose) are not small molecules, although their constituent monomers—ribo- or deoxyribonucleotides, amino acids, and monosaccharides, respectively—are considered to be. Very small oligomers are also considered small molecules, such as dinucleotides, peptides such as the antioxidant glutathione, and disaccharides such as sucrose.

“Twist-1”, also referred to as Twist1, twist1, twist-1, ACS3, BPES2, BPES3, H-twist, SCS, TWIST, bHLHa38, twist, B-HLH DNA binding protein, TWIST homolog of drosophila, acrocephalosyndactyl)-3, blepharophimosis, epicanthus inversus and ptosis 3, and twist homolog 1 (Drosophila), refers to a transcription factor which is a basic-helix-loop-helix transcription factor associated with Saethre-Chotzen syndrome. Basic helix-loop-helix (bHLH) transcription factors have been implicated in cell lineage determination and differentiation. The protein encoded by this gene is a bHLH transcription factor and shares similarity with another bHLH transcription factor, Dermo1. The strongest expression of this mRNA is in placental tissue; in adults, mesodermally derived tissues express this mRNA preferentially. Mutations in this gene have been found in patients with Saethre-Chotzen syndrome. The amino acid sequence of human Twist-1 is that of SEQ ID NO:4.

AKT protein family, which members are also called protein kinases B (PKB) plays an important role in mammalian cellular signaling. In humans, there are three genes in the “Akt family”: Akt1, Akt2, and Akt3. These genes code for enzymes that are members of the serine/threonine-specific protein kinase family (EC 2.7.11.1).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

### Materials and Methods

### Constructs:

Human Twist1 and its variants were subcloned into pcDNA3.1, pBABEpuro and pLNCX2 (Clontech) vectors. Twist1-targetting shRNA (5′-AAGCTGAGCAAGATTCAGACC-3′ (SEQ ID NO:1) (Yang et al., 2004a)) was subcloned into pSUPER.retro.puro vector. To construct the rescue plasmids expressing Twist1 and its variants, silent point mutations were introduced individually by PCR using primers 5′-TGCAAGCTTTCGAAGATTCAGACCCTCAAGC-3′ (SEQ ID NO:2) and 5′-TGCGTCGAC-ctagtgggacgcggacatgg-3′ (SEQ ID NO:3), resulting in unchanged protein sequences but resistance to shRNA-induced knockdown of Twist mRNA.

### Cell Culture and Retro Viral Infection:

The Akt1 and Akt2 double-knockout MEF cell line (MEF_Akt1/2dKO) was a gift from Dr. Morris Birnbaum, University of Pennsylvania (Liu et al., 2006). 67NR, 168FARN, 4TO7 and 4T1 cell lines were provided by Dr. Nancy Hynes (Friedrich Miescher Institute for Biomedical Research). Human glioblastoma multiform cell lines were obtained from Dr. Adrian Merlo, Sonnenhof Hospital, Bern, Switzerland. All cell lines were maintained in DMEM supplemented with 10% fetal bovine serum, 2 mM L-glutamine and 1% penicillin/streptomycin. To produce recombinant retrovirus, 20 μg of DNA was transfected into 5×10⁶ of packaging cells growing on a 10-cm plate. After 24 h, the medium was refreshed and incubated at 30° C. for 4872 h. To infect targeting cells, the supernatant containing recombinant retroviruses was filtered (0.45 μm, Millipore), mixed with 4 μg/ml of polybrene (Sigma) and placed onto freshly split overnight cultures for 6 h. After a 24-h of incubation in normal growth medium at 37° C., the infected cells were selected by appropriate antibiotics: MEF cells with 2 μg/ml puromycin, 4T1_Tw1KD cells with 12 μg/ml puromycin, MDCK cells expressing Twist1 variants with 4 μg/ml puromycin, and 4T1_Tw1KD cells expressing Twist1 variants with 5 μg/ml puromycin and 800 μg/ml G418.

### Reagents:

LY294002 was from Alexis. For western blot analysis, cells were lyzed in standard SDS-PAGE loading buffer and sonicated. For the subcellular fractionation assay, cells were resuspended in cold RSB buffer containing 10 mM HEPES, 10 mM NaCl, 1.5 mM MgCl₂, 1 mM benzamidine, 4 μM leupeptin, 0.5 mM PMSF, 1 μM microcystin and 1 mM DTT at pH 6.2. Polyclonal Twist1 and S42-phospho-specific Twist1 antibodies were previously described (Vichalkovski et al. 2010). The other antibodies against Akt1, Akt2 (Alexis), pan-Akt, Akt-pT308, Akt-pS473 and MMP-9 (Cell Signaling), LaminA/C and Acin (Santa Cruz), E-cadherin, N-cadherin, α-catenin, β-catenin, γ-catenin, Snail2 and fibronectin (BD Biosciences), vimentin V9 (Thermo Scientific), tenascin C and α-tubulin (FMI) and cytokeratin 8 (Boehringer Mannheim) were applied according to the supplier's instructions. Horseradish peroxidase-conjugated donkey anti-rabbit, sheep anti-mouse or goat-anti rat (Amersham) were used as secondary antibodies.

### Immunocytochemistry:

Cells were fixed with 4% paraformaldehyde for 20 min and permeabilized with 0.1% Triton X-100 for 10 min at room temperature. After PBS washing, cells were blocked in 3% BSA for 1 h. Individual primary antibodies were incubated at 4° C. overnight. Secondary antibodies conjugated with variable fluorphores were applied for 1 h. Cell nuclei were stained with DAPI and mounted in Vectashield (Vector Laboratories, USA).

### Immunohistochemistry:

All tissue samples were fixed in 4% paraformaldehyde and paraffin embedded. Sections (˜3 μm) were cut and processed automatically (HM355S). Staining was carried out (Vantana Discovery XT) under standard conditions. The rabbit polyclonal Ki67 was from Neomarkers and Mammaglobin was a gift from Dr. H. Hedman (Sweden).

### Clinical Breast Tumor Samples:

The use of human breast tumor samples from the Biobank (Institute of Pathology, University hospital, Basel, Switzerland) for retrospective studies was sanctioned by the ethics committee of Basel (EKBB, Switzerland). Out of a total of 2094, 1532 invasive breast tumor samples were evaluable and used in this study. The average age of the patients was 63 years. These tumors were clinically diagnosed as 59% invasive ductal carcinoma, 11% invasive lobular carcinoma and 30% other types. The tissue microarray technology was described previously (Tzankov et al., 2005).

### Cell Migration and Invasion Assays:

A wound healing assay was performed using Ibidi chambers (Ibidi GmbH, Germany). After serum starvation overnight, the cell migration was visualized and recorded (Zeiss TILLS, LONG RUN, Axiovert 200M) for 2040 h at 37° C. The recovery of wounded area was plotted and quantified using Metamorph. A Matrigel-based invasion assay was carried out using a BioCoat Matrigel invasion chamber (BD Biosciences) and performed as described previously (Clark et al., 2000). All samples were analyzed in triplicates.

### Mouse Model of Breast Cancer Metastasis:

Animal maintenance and experimental procedures conformed to the Swiss Animal Protection Ordinance. Surgery was carried out on female 12- to 15-week-old BALB/c mice purchased from Charles River Laboratories (France). Tumor cells (5×10⁵) were injected into the mammary gland of mice anesthetized by isoflurane. The size of primary tumors was measured weekly with calipers. Dissected primary tumors, the spleens and the lungs were quickly rinsed with PBS and fixed in formaldehyde for histopathologic analysis. Tumor nodules in lung were stained with Bouin solution. The numbers of visible tumor nodules in lung were subsequently counted under a dissection microscope (Leica MacroFluo Z6 Apo).

### Results

Twist1 is Phosphorylated on S42 by Activated Akt and Correlates with Enhanced Akt Phosphorylation:

It has been observed in tine inventors' laboratory that ectopically expressed Twist1 phosphorylated on S42 was frequently observed in the nuclei of both untransformed and cancer cell lines. To investigate whether translocation into the nucleus is dependent on S42 phosphorylation, the present inventors transiently expressed wild-type Twist1 and its non-phosphorylatable mutant Twist1/S42A in MDCK cells. Forty-eight hours after transfection, Twist1 was strongly phosphorylated on S42. Interestingly, this was associated with increased phosphorylation level of Akt on serine 473 (S473), whilst total Akt remained unchanged. To explore whether Twist1 phosphorylation and Akt activation are positively associated, the present inventors inhibited the PI3K/Akt pathway by overnight starvation of MDCK cells expressing Twist1 or Twist1/S42A and subsequently activated Akt by substituting normal medium containing 10% FBS for 1 h. Withdrawal of serum strongly reduced S473 phosphorylation of Akt and this corresponded with a reduction in Twist1 phosphorylation. Serum stimulation rapidly and robustly activated Akt with concomitant phosphorylation of Twist1 on S42. Consistent with the level of S473 phosphorylation, Akt phosphorylation was much greater in response to serum stimulation of MDCK cells expressing Twist1 than those expressing Twist1/S42A or an empty plasmid, showing four-fold and nine-fold higher activation, respectively. The specificity of Twist1 S42 phosphorylation by Akt was further indicated using Akt1/2-depleted MEF cells. Phosphorylation of Twist1 was not detectable in Akt1/2-depleted MEFs. Phosphorylation of Twist1 was not detected in Akt1/2-depleted MEFs under basal conditions or after insulin stimulation, but was high in stimulated wild-type MEFs. It is well documented that Akt phosphorylation of transcription factors affects their subcellular localization. Therefore the effect of S42 phosphorylation on Twist1 subcellular location was explored by immunofluorescence staining. Twist1 and Twist1/S42A both localized exclusively to the nucleus, indicating that the S42 phosphorylation is not required for nuclear translocation. However, importantly, in MDCK cells expressing wild-type Twist1, increased Akt phosphorylation was detected on cytoplasmic membranes, where it exclusively co-localized with enhanced actin bundles resulting from increased actin polymerization. This re-organization of cytoskeleton was not found in Twist1/S42A-expressing cells, suggesting a potentially important impact of Twist1 phosphorylation on cell morphogenesis and motility. As phosphorylated Akt proteins have been frequently detected in the nucleus (Shiraishi et al., 2004; Wang and Brattain, 2006), the present inventors explored whether Twist1 is preferentially phosphorylated by active Akt in the nucleus. Subcellular fractionation assays using MDCK cells expressing Twist1 or Twist1/S42A detected phosphorylated Akt proteins together with phosphorylated Twist1 in purified nuclear fractions. Moreover, consistent with the earlier observations, enhanced Akt phosphorylation was enriched in purified membrane fractions, as well as in cytoplasmic and nuclear extracts but was not observed in Twist1/42A-expressing cells.

Phosphorylation of Twist1 Triggers EMT Associated with Increased Cell Migration and Invasion:

Accompanying increased cell motility, Twist1 was shown to convert untransformed epithelial cells such as MDCK to a fibroblast-like morphology. Given the dramatic re-organization of the cytoskeleton observed in MDCK cells upon Twist1 phosphorylation, the present inventors examined whether regulation of cell migration was controlled by Twist1 phosphorylation. For this, they evaluated cell migration and invasion by live-imaging microscopy of MDCK cells expressing Twist1 or its variants. In preliminary wound-healing assays using standard DMEM medium containing 10% serum, MDCK cells recovered about 35% of the wounded area within 8 h when expressing wild-type Twist1 but only 4% when expressing Twist1/S42A. Thus, the potential of cell migration was strongly dependent on Twist1 phosphorylation. To mimic the in vivo microenvironment, a Boyden-chamber-based invasion assay was performed in the presence or absence of serum. The invasive potential of the three experimental cell lines after withdrawal of serum was maintained at basal level with no significant differences between the lines, which was consistent with former observations (Yang et al., 2004a). However, when Twist1 was rapidly phosphorylated by Akt after serum stimulation, invasion was significantly enhanced eightfold for MDCK cells expressing wild-type Twist1 compared with Twist1/S42A, suggesting a positive correlation between cell invasion and Akt phosphorylation of Twist1. As cells expressing Twist1 display morphology consistent with an EMT phenotype as well as increased migration and invasion, the affects of Twist1 phosphorylation on the expression and localization of EMT molecular markers were examined. Expression of wild-type Twist1 or Twist1/S42A significantly downregulated the adhesion molecules E-cadherin, α-, β- and γ-catenin, ZO-1 and cytokeratin 8, which are all required for the formation of intercellular junctions to maintain the apical-basal cell polarity. Cells identified by staining for E-cadherin displayed cell scattering and were elongated and spindle-shaped. However, when Twist1 alone was phosphorylated the upregulation of the mesenchymal proteins Fibronectin, Vimentin, metalloproteinase 9 (MMP-9) and Tenascin C, commonly seen during EMT, was also observed. Interestingly, a further essential EMT-driving transcription factor, Snail2, was also upregulated in Twist1-expressing cells, suggesting an additive effect ensuring a complete EMT phenotype after Twist1 phosphorylation. The contribution of the S42 phosphorylation to this process was confirmed by using phospho-mimic mutants of Twist1 with S42 changed to aspartate (Twist1/S42D) that displayed the same patterns of epithelial and mesenchymal protein expression and cell motility as wild-type Twist1. These findings indicate that the complete EMT phenotype with associated morphological changes requires PI3K/Akt activation-mediated phosphorylation on S42 of Twist1, placing this PI3K/Akt/Twist1 axis at a central position in the complete EMT program involved in cell migration and invasion.

Twist1 is Phosphorylated in Invasive Cancer Cells and Correlated with PI3K/Akt Activity:

The observation that S42 phosphorylation of Twist1 is crucial for EMT in non-transformed cells prompted the present inventors to determine whether this is also the case for EMT in tumor cells. In a previous report (Yang et al., 2004a), the expression level of Twist1 was shown to be correlated with the invasive potential of four isolated breast cancer cell lines derived from the same spontaneous murine breast tumor. Of these, the most invasive cell line, 4T1, showed the highest Twist1 expression. Orthotopic injection of 4T1 cells into the mammary fat pad of BALB/c mice resulted in dissemination of tumor cells into the bloodstream and the development after 21 days of metastases in distant organs, including lung, ovary, kidney, liver and brain (Aslakson and Miller, 1992). The present inventors compared S42 phosphorylation of Twist1 in these cell lines and human cancer cell lines of varying metastatic potential. Consistent with the proposed importance of Twist1 S42 phosphorylation in EMT, Twist1 was highly phosphorylated in metastatic 4T1 and 168FARN cells, which also metastasize to lymph nodes and form micrometastases. In contrast, the adenocarcinoma cell lines MDA-MB-481 and MDA-MB-231 and the melanoma cells MDA-MB-435 showed no phosphorylation on S42 despite moderate expression of Twist1. Similarly, although its expression has been frequently detected (Gort et al., 2008), endogenous Twist1 was not phosphorylated in HeLa cells. MCF7 cells, as a negative control, do not express Twist mRNA and Twist activity was not detected. As Twist1 was also reported to be highly expressed in invasive human gliomas and to promote cancer invasion (Elias et al., 2005), and as brain tissue microarray analysis (Korur et al., 2009) revealed elevated Twist1 in malignant human glioblastoma multiforme (GBM), Twist1 status was assessed in several clinically classified invasive glioblastoma cell lines. The present inventors found Twist1 to be phosphorylated in several such lines, e.g., BS125 and U373MG. To examine whether endogenous Twist1 phosphorylation is reduced after inhibition of the PI3K/Akt pathway, they treated 4T1 cells with the widely used PI3K/Akt inhibitor LY294002. Akt phosphorylation on S473 declined after 30 min incubation with LY294002 and Twist1 phosphorylation was also strongly inhibited, similar to their earlier observations in MDCK cells expressing Twist1. 4T1 cell motility assessed by incubating cells in serum-free medium was significantly reduced, an effect that was rapidly reversed by addition of 10% serum. This serum-stimulated migration was reduced to the basal level by addition of LY294002 to the medium. These results indicate that the cell motility important for invasive activity of 4T1 cells requires Twist1 phosphorylation mediated by PI3K/Akt.

Phosphorylation of Twist1 is Essential for Breast Tumor Metastasis from the Mammary Gland to the Lung:

Knockdown of Twist1 in 4T1 cells (4T1_Tw1KD) dramatically abolished the growth of metastatic tumor nodules in lung in the sygeneic xenograft breast tumor model (Yang et al., 2004a). To explore whether Twist1-promoted lung metastasis is dependent on S42 phosphorylation, the present inventors generated several rescue clones expressing Twist1 and its variants in 4T1_Tw1KD cells. Consistent with the established role of Twist1 in the downregulation of E-cadherin, knockdown of Twist1 significantly restored the E-cadherin in 4T1 cells, mediating the re-establishment of adhesion junctions and cell-cell contacts. After renewed expression of exogenous Twist1 in these cells, S42 phosphorylation of wild-type Twist1 occurred and intercellular contacts were again disrupted. However, as found with the re-expression of the exogenous Twist/S42A, this did not seem to require phosphorylation of S42, consistent with the observations in MDCK cells. Cells re-expressing Twist1 or its mutants were injected into the mammary fat pad of 12- to 15-week-old BALB/c mice. After 21 days, knockdown of Twist1 had not affected primary tumor formation, compared with the injection of either parental 4T1 cells or 4T1_Tw1KD cells expressing Twist1 variants. Thus, Twist1 did not have a substantial impact on primary tumor growth in the 4T1 mouse model. Furthermore, compared with control-injected BALB/c mice, splenomegaly (DuPre and Hunter, 2007) occurred in all rescue clones despite Twist1 depletion, indicating that the leukoid reaction to colony-stimulating factors in 4T1 tumor-bearing mice did not depend on Twist1 expression. However, when ectopic Twist1 was phosphorylated in 4T1_Tw1KD cells, metastatic tumor nodules in lung were significantly restored, whereas injection of 4T1_Tw1KD/Twist1/S42A cells rarely produced visible tumor nodules. In contrast to the S42A mutant, phospho-mimic S42D cells robustly induced lung metastases similar to wild-type Twist1. These data demonstrate that Akt-mediated S42 phosphorylation is critical for Twist1-triggered lung metastases. To determine whether the observed metastatic tumors originated from injected 4T1 cells, the present inventors performed immunohistochemical staining of the lung tissues for mammary cell markers. Haematoxylin and Eosin (H/E) staining identified a random distribution of solid nodules of various sizes and irregular margins. These lesions were highly proliferative compared with the adjacent normal lung tissues. Correspondingly, these regions were characterized by prominent expression of mammaglobin, a mammary epithelium-specific protein overexpressed in breast cancers (Sasaki et al., 2007; Watson et al., 1999) that is one of the most frequently used diagnostic markers in clinics to verify the breast origin of recurrent tumors (Bhargava et al., 2007; Sasaki et al., 2007; Watson et al., 1999). Consistently, the ratio of mammaglobin and Ki67 positive cells in lungs was significantly higher in mice injected with 4T1_Tw1KD/Twist1 or 4T1_Tw1KD/Twist1/S42D. This staining pattern clearly confirmed that the lung tumor nodules originated from injected mammary epithelium 4T1_Tw1KD cells expressing Twist1 variants. However, Twist1 was not required for the formation of primary tumor. These data indicate that Akt-mediated Twist1 phosphorylation influences lung metastasis via enhanced cell invasiveness.

### Twist1 is Ubiquitously Phosphorylated in Invasive Human Breast Cancers:

As overexpression of Twist1 is frequent in many different human cancers and high Twist1 expression was detected in 70% ILC from 57 invasive human breast tumors analyzed (Yang et al., 2004a), the present inventors examined whether Twist1 phosphorylation was correlated with the clinical pathological features of the invasive breast cancers, taking advantage of high-throughput tissue microarray examination of total 2094 invasive human breast cancer samples (Tzankov et al., 2005). Strikingly, in 1532 tumor samples that are evaluable, Twist1 phosphorylation was detected in more than 90% of the breast cancer samples, correlated with high phosphorylation level of Akt phosphorylation. Interestingly, in contrast to the previous analysis of Twist1 upregulation in ILC (Yang et al., 2004a), the present inventors' data showed that Twist1 was highly phosphorylated and not significantly different in clinically diagnosed invasive ductal carcinomas (IDC), ILC and mixed IDC/ILC, at 96.2%, 94.7% and 94.4%, respectively. This strongly suggests that Twist1 phosphorylation is a ubiquitous process in invasive breast cancers.

### REFERENCES

Ansieau, S., et al. (2008). Cancer Cell 14, 79-89. Arboleda, M. J., et al. (2003). Cancer Res 63, 196-206. Aslakson, C. J., and Miller, F. R. (1992). Cancer Res 52, 1399-1405. Bhargava, R., Beriwal, S., and Dabbs, D. J. (2007). Am J Clin Pathol 127, 103-113. Brazil, D. P., Yang, Z. Z., and Hemmings, B. A. (2004). Trends Biochem Sci 29, 233-242. Castanon, I., and Baylies, M. K. (2002). Gene 287, 11-22. Chalhoub, N., and Baker, S. J. (2009). Annu Rev Pathol 4, 127-150. Chen, Z. F., and Behringer, R. R. (1995). Genes Dev 9, 686-699. Cheng, G. Z., et al. (2007). Cancer Res 67, 1979-1987. Christiansen, J. J., and Rajasekaran, A. K. (2006). Cancer Res 66, 8319-8326. Clark, E. A., et al. (2000). Nature 406, 532-535. Dillon, R. L., et al. (2009). Cancer Res 69, 5057-5064. DuPre, S. A., and Hunter, K. W., Jr. (2007). Exp Mol Pathol 82, 12-24. Elias, M. C., et al. (2005). Neoplasia 7, 824-837. Engelman, J. A. (2009). Nat Rev Cancer 9, 550-562. Fayard, E., et al. Curr Top Microbiol Immunol. Garber, K. (2008). J Natl Cancer Inst 100, 232-233, 239. Geiger, T. R., and Peeper, D. S. (2009). Biochim Biophys Acta 1796, 293-308. Gort, E. H., et al. (2008). Cancer Epidemiol Biomarkers Prev 17, 3325-3330. Grunert, S., Jechlinger, M., and Beug, H. (2003). Nat Rev Mol Cell Biol 4, 657-665. Hutchinson, J. N., et al. (2004). Cancer Res 64, 3171-3178. Iliopoulos, D., et al. (2009). Sci Signal 2, ra62. Irie, H. Y., et al. (2005). J Cell Biol 171, 1023-1034. Ju, X., et al. (2007). Proc Natl Acad Sci USA 104, 7438-7443. Kang, Y., and Massague, J. (2004). Cell 118, 277-279. Klymkowsky, M. W., and Savagner, P. (2009). Am J Pathol 174, 1588-1593. Korur, S., et al. (2009). PLoS One 4, e7443. Larue, L., and Bellacosa, A. (2005). Oncogene 24, 7443-7454. Lee, J. M., et al. (2006a). J Cell Biol 172, 973-981. Lee, T. K., et al. (2006b). Clin Cancer Res 12, 5369-5376. Liu, P., et al. (2009). Nat Rev Drug Discov 8, 627-644. Liu, X., et al. (2006). J Biol Chem 281, 31380-31388. Maestro, R., et al. (1999). Genes Dev 13, 2207-2217. Maroulakou, I. G., et al. (2007). Cancer Res 67, 167-177. Mattila, P. K., and Lappalainen, P. (2008). Nat Rev Mol Cell Biol 9, 446-454. Onder, T. T., et al. (2008). Cancer Res 68, 3645-3654. Perez-Tenorio, G., and Stal, O. (2002). Br J Cancer 86, 540-545. Polyak, K., and Weinberg, R. A. (2009). Nat Rev Cancer 9, 265-273. Puisieux, A., Valsesia-Wittmann, S., and Ansieau, S. (2006). Br J Cancer 94, 13-17. Sasaki, E., et al. (2007). Mod Pathol 20, 208-214. Scheid, M. P., and Woodgett, J. R. (2001). Nat Rev Mol Cell Biol 2, 760-768. Shiraishi, I., et al. (2004). Circ Res 94, 884-891. Stasinopoulos, I. A., et al. (2005). J Biol Chem 280, 2294-2299. Tarin, D., Thompson, E. W., and Newgreen, D. F. (2005). Cancer Res 65, 5996-6000; discussion 6000-5991. Thiery, J. P., et al. (2009). Cell 139, 871-890. Thiery, J. P., and Sleeman, J. P. (2006). Nat Rev Mol Cell Biol 7, 131-142. Thompson, E. W., Newgreen, D. F., and Tarin, D. (2005). Cancer Res 65, 5991-5995; discussion 5995. Tomaskovic-Crook, E., Thompson, E. W., and Thiery, J. P. (2009). Breast Cancer Res 11, 213. Trimboli, A. J., et al. (2008). Cancer Res 68, 937-945. Tzankov, A., et al. (2005). Exp Gerontol 40, 737-744. Valsesia-Wittmann, S., et al. (2004). Cancer Cell 6, 625-630. Vesuna, F., et al. (2008). Biochem Biophys Res Commun 367, 235-241. Vichalkovski A. et al. (2010). Oncogene 29, 3554-3565. Vivanco, I., and Sawyers, C. L. (2002). Nat Rev Cancer 2, 489-501. Wang, R., and Brattain, M. G. (2006). Cell Signal 18, 1722-1731. Watson, M. A., et al. (1999). Cancer Res 59, 3028-3031. Weigelt, B., Peterse, J. L., and van 't Veer, L. J. (2005). Nat Rev Cancer 5, 591-602. Yang, J., et al. (2004a). Cell 117, 927-939. Yang, J., and Weinberg, R. A. (2008). Dev Cell 14, 818-829. Yang, Z. Z., et al. (2004b). Biochem Soc Trans 32, 350-354. Yilmaz, M., and Christofori, G. (2009). Cancer Metastasis Rev 28, 15-33. Yin, G., et al. Oncogene 29, 3545-3553.

1. A method for inhibiting metastasis in a subject by modulating the phosphorylation of the Serine 42 of Twist1 by administering to said subject a therapeutically effective amount of a modulator of said phosphorylation of the Serine 42 of Twist1.

2. The method of claim 1 wherein the phosphorylation of the Serine 42 of Twist1 is modulated by an inhibitor which specifically binds to Twist1 and hinders the phosphorylation of its Serine 42 by PKB.

3. The method of claim 2 wherein the inhibitor is an antibody or a small molecule.

4. The method of claim 1 wherein the modulator is a peptide comprising an amino acid corresponding to the Serine 42 of Twist1, which fragment is recognized and phosphorylated by PKB at said amino acid corresponding to the Serine 42 of Twist1.

5. The method of claim 1 wherein the subject is a mammal.

6. The method of claim 1 wherein the epithelial-mesenchymal transition of cancer cells is reduced.

7. The method of claim 6 wherein the cancer cells are from a colorectal cancer, a breast cancer, a lung cancer or a prostate cancer.

8. (canceled)

9. (canceled)

10. A method for the identification of a substance that modulates the formation of metastasis, which method comprises the step of assessing the phosphorylation of the Serine 42 of Twist1.

11. A method of diagnosing metastasis comprising the step of assessing the phosphorylation of the Serine 42 of Twist1.

12. The method of claim 11, wherein an increased phosphorylation of the Serine 42 of Twist1 is indicative of metastasis formation.

13. (canceled)

14. The method of claim 5, wherein the mammal is a human subject.


SEQUENCE LISTING (4 sequences)

SEQ ID NO: 1 - DNA, 21 bp, Homo sapiens
aagctgagcaagattcagacc

SEQ ID NO: 2 - DNA, 31 bp, Homo sapiens
tgcaagctttcgaagattcagaccctcaagc

SEQ ID NO: 3 - DNA, 29 bp, Homo sapiens
tgcgtcgacctagtgggacgcggacatgg

SEQ ID NO: 4 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH